(19) United States
(12) Patent Application Publication
(10) Pub. No.: US 2014/0200655 A1
(43) Pub. Date: Jul. 17, 2014

(54) INTRAVASCULAR CARDIAC RESTRAINING IMPLANTS AND METHODS FOR TREATING HEART FAILURE

(71) Applicant: Abbott Cardiovascular Systems, Inc., Santa Clara, CA (US)

(72) Inventors: William E. Webler, Jr., San Jose, CA (US); Randolf von Oepen, Aptos, CA (US)

(73) Assignee: Abbott Cardiovascular Systems, Inc., Santa Clara, CA (US)

(21) Appl. No.: 13/744,216

(22) Filed: Jan. 17, 2013

(51) Int. Cl. A61F 2/848; A61F 2/82

(52) U.S. Cl. CPC A61F 2/848; A61F 2/82
USPC 623/1.18; 623/1.1; 623/1.2; 606/200; 623/1.38

(57) ABSTRACT

Intravascular cardiac restraining implants designed for treatment of heart disease and heart failure and methods for their use. The disclosed implants can be used to reshape or reinforce a diseased, weakened or distended portion of a patient's heart to counteract heart disease and heart damage. An intravascular cardiac restraining implant may include a first tissue anchor configured for implantation in a first region of a coronary vein, a second tissue anchor configured for implantation in a second region of the coronary vein, and at least one elongate member coupled to the first tissue anchor and the second tissue anchor. In one embodiment, the at least one elongate member may be a spring or a similar device configured for biasing the first and second tissue anchors toward one another, thus reshaping or reinforcing a diseased, weakened or distended portion of a patient's heart.

### BACKGROUND

1. The Field of the Invention

The present invention relates to intravascular implants configured to be deliverable and deployable percutaneously for treatment of heart failure.

2. The Relevant Technology

Congestive heart failure is a condition that can result in the inability of the heart to fill with blood or pump blood effectively. Unfortunately, there are no treatments that are currently known to be consistently effective. Many times the progression of the disease can be slowed through lifestyle changes and pharmacological management, but when unchecked, the disease can progress to the need for a full heart transplant or the patient may die.

In a specific manifestation of congestive heart failure includes the weakening of a heart region, in which the myocardium in this region will distend from more healthy heart tissue and will not contract or will only contract weakly. This distention can further inhibit proper and effective heart function and can further the disease symptoms.

Congestive heart failure often leads to a condition called megalocarida where the heart becomes enlarged as the heart muscle tries to compensate for poor heart function and poor oxygenation of the blood. Megalocardia is generally quite detrimental. For example, enlargement of the heart can cause the annular size of the heart valves that separate the atria from the ventricles to also become enlarged causing the valves to fail to close properly and blood leakage between the chambers of the heart, which further reduces cardiac function and exacerbates the tendency of the heart to enlarge in an effort to compensate for poor function. This reduction in blood flow can be life threatening, especially in patients that have lost ventricular tissue (e.g., heart attack victims), have contraction synchronization problems and/or other problems that reduce the heart's ability to act as a pump.

Myocardial infarction (i.e., heart attack) can lead to loss of heart function and morphological changes in the heart through loss of heart muscle (i.e., tissue necrosis). The dead or damaged can distend or bulge away from healthy heart tissue, further reducing cardiac function. Over time, scar tissue can replace the necrotic tissue and reinforce the heart, but it may be important to reinforce the heart tissue to prevent further damage (e.g., distension or enlargement) to the heart while waiting for scar tissue to form.

In some cases, the distention and/or enlargement of the heart can be corrected surgically. One treatment option referred to as the Batista procedure involves dissecting the heart and removing portions of the heart in order to reduce heart volume. This is a radical procedure subject to substantial controversy. Furthermore, the procedure is highly invasive, risky and expensive and commonly includes other expensive procedures (such as a concurrent heart valve replacement). If the procedure fails, emergency heart transplant is the only available option. Another treatment option pioneered by Acorn Cardiovascular, Inc. (see, e.g., U.S. Pat. No. 6,537,203) involves placing a jacket (e.g., an elastic jacket) over the heart to reshape the weakened heart, increase pumping efficiency, increase valvular efficiency, and reduce the tendency of the heart to enlarge.

However, the above described treatments are typically major surgical procedures that require the opening of the chest by sternotomy or, at best, through small incisions in the chest wall, performing a heart lung bypass and stopping the heart. While surgical procedures such as those mentioned can successfully reconstruct or reshape the heart and counteract the effects of heart disease (e.g., chronic heart failure), these problems are often associated other debilitating diseases and, thus, patients are often unable to tolerate the required open heart surgery. Therefore, there is a need for a less invasive and traumatic way to treat heart failure and enlargement of the heart.

### BRIEF SUMMARY

Described herein are intraluminal devices designed for treatment of heart disease and heart failure and methods for their use. For instance, the devices disclosed herein can be used to reshape or reinforce a diseased, weakened or distended portion of a patient's heart to counteract the effects of one or more of congestive heart failure, tissue necrosis following myocardial infarction, megalocardia (i.e., enlargement of the heart), and the like. In one embodiment, an intraluminal device includes an intravascular cardiac restraining implant. An intravascular cardiac restraining implant may include a first tissue anchor configured for implantation in a first region of a coronary vein, a second tissue anchor configured for implantation in a second region of the coronary vein, and at least one elongate member coupled to the first tissue anchor and the second tissue anchor. In one embodiment, the at least one elongate member may be a spring or a similar device configured for biasing the first and second tissue anchors toward one another, thus reshaping or reinforcing a diseased, weakened or distended portion of a patient's heart. Additionally, various spacers, braces, sleeves, or other implant features may be included.

In one embodiment, a method for treating a diseased, weakened or distended portion of a patient's heart is disclosed. The method includes (1) accessing a coronary vein of the patient's heart percutaneously, (2) positioning an intravascular cardiac restraining implant across at least a portion of the diseased, weakened or distended portion of the patient's heart via the coronary vein, and (3) deploying the intravascular cardiac restraining implant in the coronary vein of the patient's heart for reinforcing or reshaping the diseased, weakened or distended portion of the patient's heart. In one embodiment, the coronary vein includes a coronary sinus.

In another embodiment, a method for treating a diseased heart is disclosed. The method includes (1) providing an intravascular cardiac restraining implant that includes: (a) a first tissue anchor configured for implantation in a first region of a coronary vein, (b) a second tissue anchor configured for implantation in a second region of the coronary vein, and (c) at least one elongate member coupled to the first tissue anchor and the second anchor, wherein the intravascular cardiac restraining implant has a size and curvature selected to allow the medical device to conform to a size and curvature of a portion of the diseased heart. The method further includes (2) percutaneously delivering the implant to a weakened portion of the diseased heart, and (3) anchoring the first tissue and second tissue anchors in a coronary vein such that the first and second tissue anchors and the at least one elongate member span the weakened portion of the heart for remodeling the heart.

In yet another embodiment, a method is disclosed for treating heart failure by providing a support for a diseased, weakened, distended or misshapen portion of a patient's heart. The method includes, (1) percutaneously positioning an intravascular cardiac restraining implant in a coronary vein across at least a portion of the diseased, weakened, distended or misshapen portion of the patient's heart, wherein the intravascular cardiac restraining implant includes: (a) a first tissue anchor configured for implantation in a first region of the cardiac vein, (b) a second tissue anchor configured for implantation in a second region of the cardiac vein (c) at least one elongate member coupled to the first tissue anchor and the second anchor, and (d) at least one of the first tissue anchor or the second tissue anchor having at least one protruding member extending from one side of the intravascular cardiac restraining implant. The method further includes (2) deploying the intravascular cardiac restraining implant in the coronary vein of the patient's heart for reinforcing or reshaping the diseased, weakened, distended or misshapen portion of the patient's heart, and (3) piercing the coronary vein with the at least one protruding member and anchoring the protruding member into a heart muscle or connective tissue portion adjacent to the coronary vein.

These and other embodiments and features of the present invention will become more fully apparent from the following description, drawings, and/or appended claims, or may be learned by the practice of the invention as set forth hereinafter.

### BRIEF DESCRIPTION OF THE DRAWINGS

To further clarify the above and other advantages and features of the present disclosure, a more particular description of the disclosed embodiments will be rendered by reference to specific embodiments thereof which are illustrated in the appended drawings. It is appreciated that these drawings depict only typical embodiments of the disclosure and are therefore not to be considered limiting of its scope. The disclosed implants and methods for their use will be described and explained with additional specificity and detail through the use of the accompanying drawings, in which:

FIGS. 1A-1C include side views of an embodiment of an exemplary implant in a delivery or collapsed configuration, which is then expanded so as to provide a cardiac restraining or reshaping function.

FIG. 2A illustrates a flattened view of the interior surface of a stent-like intravascular cardiac restraining implant.

FIG. 2B illustrates a flattened view of the interior surface of another stent-like intravascular cardiac restraining implant.

FIGS. 2C is an end view of a stent-like intravascular cardiac restraining implant.

2D and 2E are perspective views illustrating different embodiments intravascular cardiac restraining implants.

FIGS. 2F and 2G illustrate different embodiments of protruding members t can be used to anchor intravascular cardiac restraining implants into a tissue adjacent to a site of implantation.

FIGS. 3A-3D are side views illustrating different embodiments of tissue cinching members of an intravascular cardiac restraining implant.

FIGS. 4A-4C are side views illustrating an embodiment of an intravascular cardiac restraining implant and methods of deploying such an intravascular cardiac restraining implant into a body lumen in accordance with the present invention.

FIGS. 5A-5C are a perspective view, longitudinal side and partial cross sectional views of an embodiment of an intravascular cardiac restraining implant deployed within a vein of the heart.

### DETAILED DESCRIPTION

### I. Introduction

Described herein are intraluminal devices (e.g., intravascular endoprostheses) designed for treatment of heart disease and heart failure and methods for their use. For instance, the devices disclosed herein can be configured to anchor into two different portions of a coronary vein to reshape or reinforce a diseased, weakened or distended portion of the heart. Such reshaping or reinforcement of the tissues of the heart can counteract the effects of one or more of congestive heart failure, tissue necrosis following myocardial infarction, megalocardia (i.e., enlargement of the heart), and the like.

In one embodiment, an intraluminal device includes an intravascular cardiac restraining implant. An intravascular cardiac restraining implant may include a first tissue anchor configured for implantation in a first region of a coronary vein, a second tissue anchor configured for implantation in a second region of the coronary vein, and at least one elongate member coupled to the first tissue anchor and the second tissue anchor.

In one embodiment, the at least one elongate member may be a substantially rigid bar, rod, or the like that is configured to apply a restraining load to the tissues of the heart. In another embodiment, the at least one elongate member may be a spring or a similar device configured for biasing the first and second tissue anchors toward one another to provide a cinching load to the tissues of the heart. The biasing member can be in an elongated length or a contracted length, when in the elongated length the biasing member(s) may automatically attempt to return to the contracted length. The attempted contraction may be instantaneous after implantation, initiated by removal of a brace holding the cinching members in the elongated length, or time delayed after biodegradation of a bracing spacer.

### II. Intravascular Cardiac Restraining Implants

The Figures described herein illustrate various embodiments of an intravascular cardiac restraining implant that includes two tissue anchors coupled together by one or more elongate members. The illustrated embodiments of the implants as well as anchors and elongate members can be combined and interchanged. While the intravascular cardiac restraining implants are shown with the same type of anchor, different types of anchors can be positioned at opposite ends of the elongate member(s). Also, the embodiments and features of each Figure and the accompanying descriptions can be combined with or substituted into embodiments and features of other Figures.

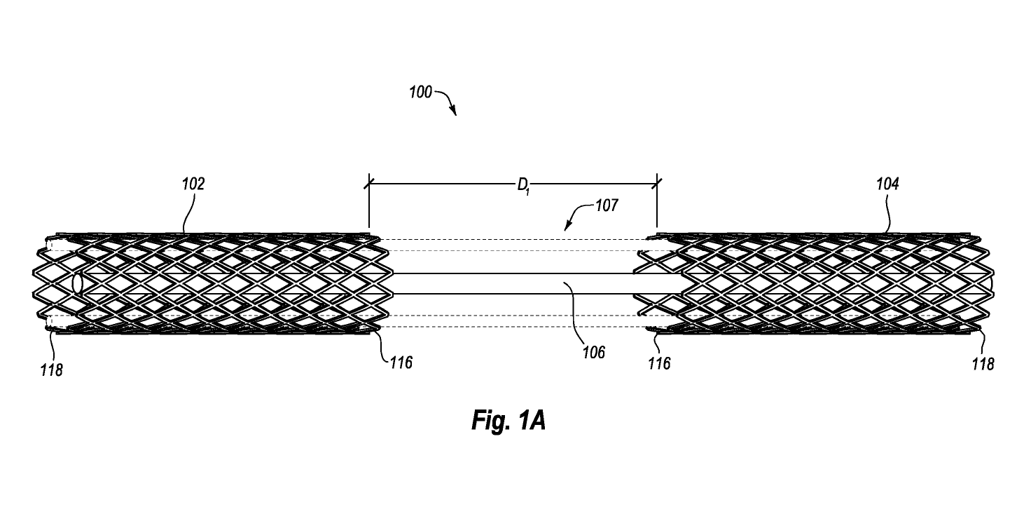

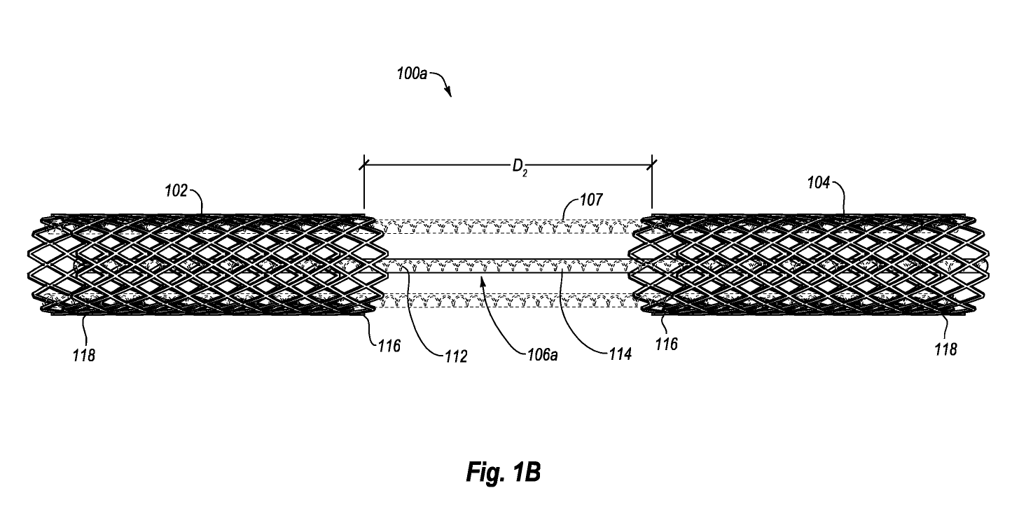

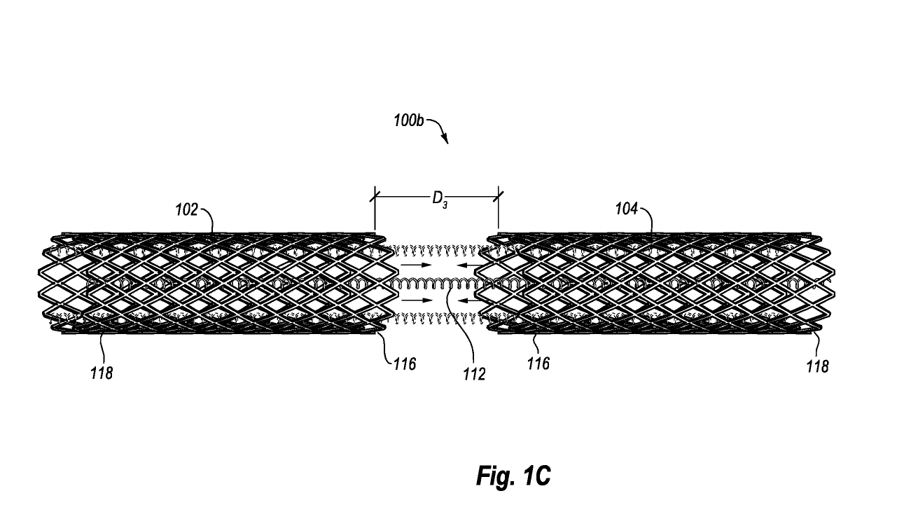

FIGS. 1A-1C show an embodiment of an intravascular cardiac restraining implant 100 having a first tissue anchor 102 connected to a second tissue anchor 104 through at least one elongate member 106. Each of the first tissue anchor 102 and the second tissue anchor 104 having an interior end 116 and an exterior end 118. As shown in the illustrated embodiment, the interior ends 116 are oriented toward one another and the exterior ends 118 are oriented away from one another. In the illustrated embodiment, the at least one elongate member 106 extends substantially from the exterior end 118 of the first tissue anchor 102 to the exterior end 118 of the second tissue anchor 104. Extending the at least one elongate member 106 substantially from the exterior end 118 of the first tissue anchor 102 to the exterior end 118 of the second tissue anchor 104 can provide the intravascular cardiac restraining implant 100 with greater flexural and/or torsional rigidity so that the implant 100 is better able to reinforce and/or reshape tissues of the heart. Optionally, the intravascular cardiac restraining implant 100 can include at least a second elongate member 107 that also extends substantially from the exterior end 110a of the first tissue anchor 102 to the exterior end 110b of the second tissue anchor 104.

In one embodiment, the elongate members (e.g., members 106 and 107) are substantially longitudinally rigid. Such rigid elongate members can act to reinforce diseased or weakened tissues of the heart by acting to resisting bulging or enlargement of the heart. Such rigid elongate members can also act to resist bulging or enlargement of the heart by providing a rigid or semi-rigid reinforcing member in the heart that the muscle can work against. In another embodiment, at least one of the elongate members (e.g., members 106 and 107) can be a spring or a similar contractile member that can reinforce or reshape diseased or damaged tissue of the heart by retracting to draw tissues in the region of the first and second tissue anchors 102 and 104 toward one another. Optionally, the intravascular cardiac restraining implant 100 may include one or more extension spacers and/or brace spacers (not shown) removably braced between the first anchor 102 and second anchor 104 so as to hold the anchors apart and/or elongate the elongate member 106.

FIG. 1A shows the intravascular cardiac restraining implant 100 in a delivery conformation that is collapsed and compact so as to be capable of being retained within a delivery catheter for delivery through or to a body lumen. The delivery conformation can have the at least one elongate member 106 elongated such that the first and second tissue anchors 102 and 104 are separated by a longitudinal dimension D₁.

FIG. 1B shows the intravascular cardiac restraining implant 100a as the first tissue anchor 102 (in the form of an expandable anchor, such as a stent) and second tissue anchor 104 have expanded so as to be capable of being anchored into a body lumen. Optional brace spacer(s) (not shown) can be removed to allow the at least one elongate member 106a to begin applying a cinching force to the first anchor 102 and second anchor 104. The cinching force can cinch the first anchor 102 and second anchor 104 together such that the first and second tissue anchors 102 and 104 are separated by a longitudinal dimension D₂ that is shorter than D₁.

Also shown in FIG. 1B is the at least one elongate member 106a being formed of a tension member 112 and a non-tension member 114. The tension member 112 is configured to apply the tension to the anchors 102 and 104 so as to provide the cinching force. The non-tension member 114 is configured to inhibit the tension member 112 from applying the cinching force to the anchors 102, 104. The non-tension member 114 can be configured to function similarly to the optional brace spacer to maintain a selected separation between the anchors 102 and 104 and be removably coupled with the tension member 112. The non-tension member 114 can be removed or separated from the tension member 112 to facilitate cinching the anchors 102, 104, or allowed to be removed or degraded. In the instance the non-tension member 114 is degradable, such as through use of a suitable biodegradable material; the tension member 112 can be freed from the non-tension member 114 over time such that the cinching force increases over time. The degradation rate of the non-tension member 114 can be designed for a particular rate of increased cinching force applied to the anchors 102, 104 by the tension member 112. The illustrated embodiment of the tension member 112 is configured as a coil spring, and the illustrated embodiment of the non-tension member 114 is configured as a biodegradable sleeve or coating that fills in the interstitial space between the coils of the spring and inhibits the spring from contracting.

FIG. 1C shows the intravascular cardiac restraining implant 100b after the optional brace and/or the non-tension member 114 has been removed or allowed to degrade so as to allow the tension member 112 to apply the cinching force (shown by the medially-oriented arrows) to the anchors 102 and 104. The cinching member 112 can have a shortened longitudinal dimension of D₃ that is shorter than D₂.

Alternatively, the longitudinal dimension D₁ may stay substantially the same over the course of the implantation; however, the at least one elongate member 106 and/or tension member 112 can apply the cinching force to the tissues to hold them in place and provide support rather than drawing the anchors 102 and 104 together.

The intravascular cardiac restraining implants of the present invention can be made of a variety of materials, such as, but not limited to, those materials which are well known in the art of implant manufacturing. This can include, but not limited to, an implant having a primary material for at least one of the anchors and/or the elongate members that join the anchors. The anchors and/or elongate members can each be prepared from a primary material as its core or substrate, and include layers of polymer or metallic layers to provide additional features to the anchors. Generally, the materials for the implant can be selected according to the structural performance and biological configurations that are desired.

In one configuration, the elongate members and/or the anchors have multiple layers, with at least one layer being applied to a primary material or substrate forming the core of the anchors. As such, the anchor can have multiple layers that are different from one another. The multiple layers on the elongate members and/or the anchors can be resiliently flexible materials or rigid and inflexible materials. For example, one layer can be a coating that is applied over the entire intravascular cardiac restraining implant, or to select portions. The select portions can include the layer of polymer being applied over the couplings, elongate member, anchors or other portion

For example, materials such as Ti3Al2.5V (also referred to as 3-2.5Ti), Ti6Al4V (also referred to as 6-4Ti), Ti6Al7Nb, Ti6AlV, and platinum may be particularly good choices for adhering to a flexible material, such as, but not limited to, Nitinol. The use of resiliently flexible materials can provide cinching or shortening forces to the anchors upon being stretched. The use of resiliently flexible elongate members and/or brace spacers, which can also be beneficial for absorbing stress and strains. Also, the multiple layers can be useful for applying radiopaque materials to the anchors. For example, types of materials that are used to make an implant can be selected so that the implant is capable of being collapsed during placement or delivery and expanded when deployed. Usually, the implant can be self-expanding, balloon-expandable, or can use some other well-known configuration for deployment. For purposes of illustration and not limitation, reference is made generally to self-expanding embodiments and balloon-expandable embodiments of the implant of the present invention; however, other types of implants can be configured in accordance with the present invention.

Various different manufacturing techniques are well known and may be used for fabrication of the intravascular cardiac restraining implant of the present invention. Such manufacturing techniques can be employed to make the different anchors or spacers of the intravascular cardiac restraining implant. For example, the different anchors or spacers can be formed from a hollow tube using a known technique, such as laser cutting, EDM, milling, chemical etching, hydro-cutting, and the like. Also, the different anchors or spacers can be prepared to include multiple layers or coatings deposited through a cladding process such as vapor deposition, electroplating, spraying, or similar processes. Also, various other processes can be used such as those described below and or others known to those skilled in the art in light of the teaching contained herein.

Optionally, the anchors can be fabricated from a sheet of suitable material, where the sheet is rolled or bent about a longitudinal axis into the desired tubular shape. Additionally, either before or after being rolled into a tube, the material can be shaped to include anchor features such as having stent, filter, or other medical device features. Also, the spacers can be shaped into a spacer in accordance with the descriptions of the properties of the spacers. The anchors and spacers can be shaped by well-known techniques such as laser-cutting, milling, etching or the like. The edges of the anchors and spacers can be joined together, such as by welding or bonding.

The implant (i.e., the tissue anchors and/or the elongate members) can include a coating or spacer made from a biodegradable or bioabsorbable materials, which can be either plastically deformable or capable of being set in the deployed configuration. If plastically deformable, the material can be selected to allow the implant to be expanded in a similar manner using an expandable member so as to have sufficient radial strength and scaffolding and also to minimize recoil once expanded. If the polymer is to be set in the deployed configuration, the expandable member can be provided with a heat source or infusion ports to provide the required catalyst to set or cure the polymer.

In one embodiment, the substrate or core of the anchors and/or spacers can be prepared from a biocompatible polymer. Examples of such biocompatible polymers can include a suitable hydrogel, hydrophilic polymer, biodegradable polymers, bioabsorbable polymers. Examples of such polymers can include nylons, poly(alpha-hydroxy esters), polylactic acids, polylactides, poly-L-lactide, poly-DL-lactide, poly-L-lactide-co-DL-lactide, polyglycolic acids, polyglycolide, polylactic-co-glycolic acids, polyglycolide-co-lactide, polyglycolide-co-DL-lactide, polyglycolide-co-L-lactide, polyanhydrides, polyanhydride-co-imides, polyesters, polyorthoesters, polycaprolactones, polyesters, polyanydrides, polyphosphazenes, polyester amides, polyester urethanes, polycarbonates, polytrimethylene carbonates, polyglycolide-co-trimethylene carbonates, poly(PBA-carbonates), polyfumarates, polypropylene fumarate, poly(p-dioxanone), polyhydroxyalkanoates, polyamino acids, poly-L-tyrosines, poly(beta-hydroxybutyrate), polyhydroxybutyrate-hydroxyvaleric acids, combinations thereof, or the like.

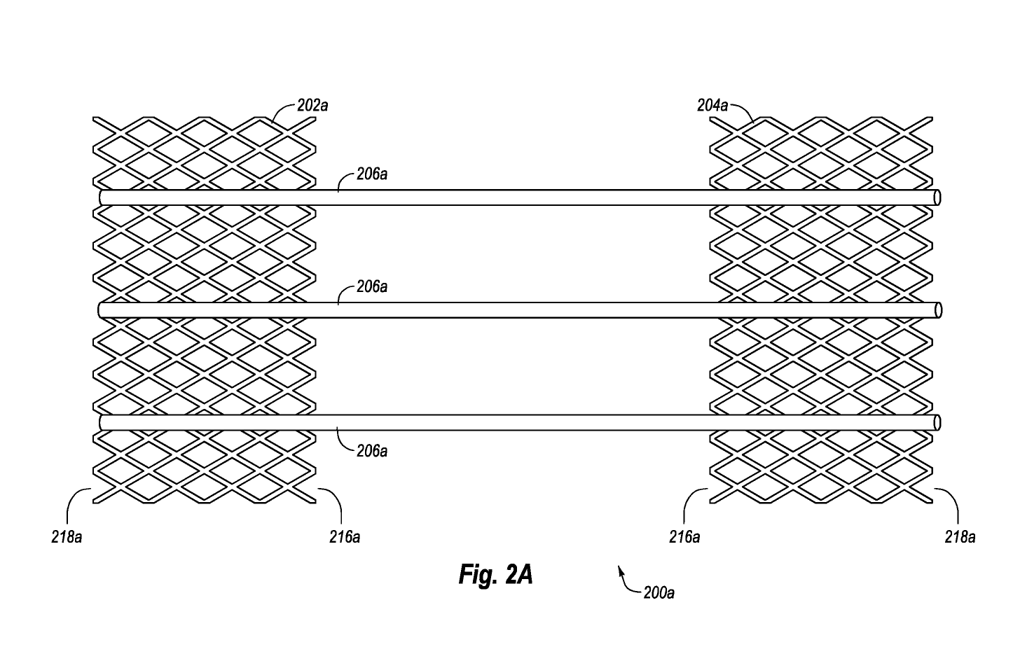

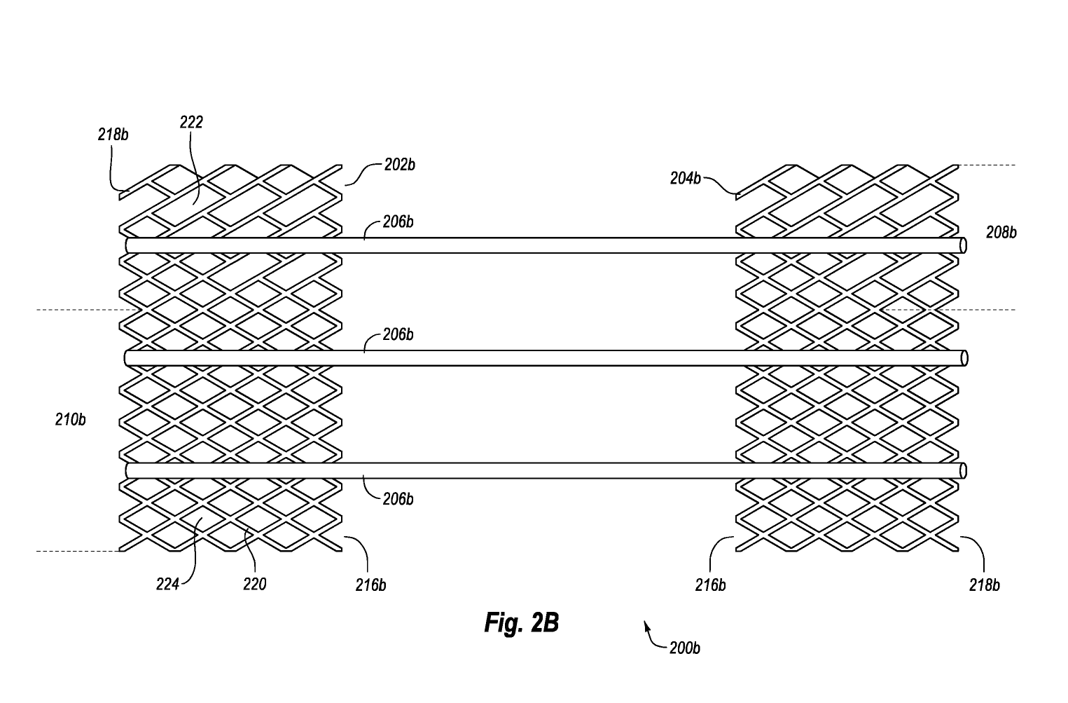

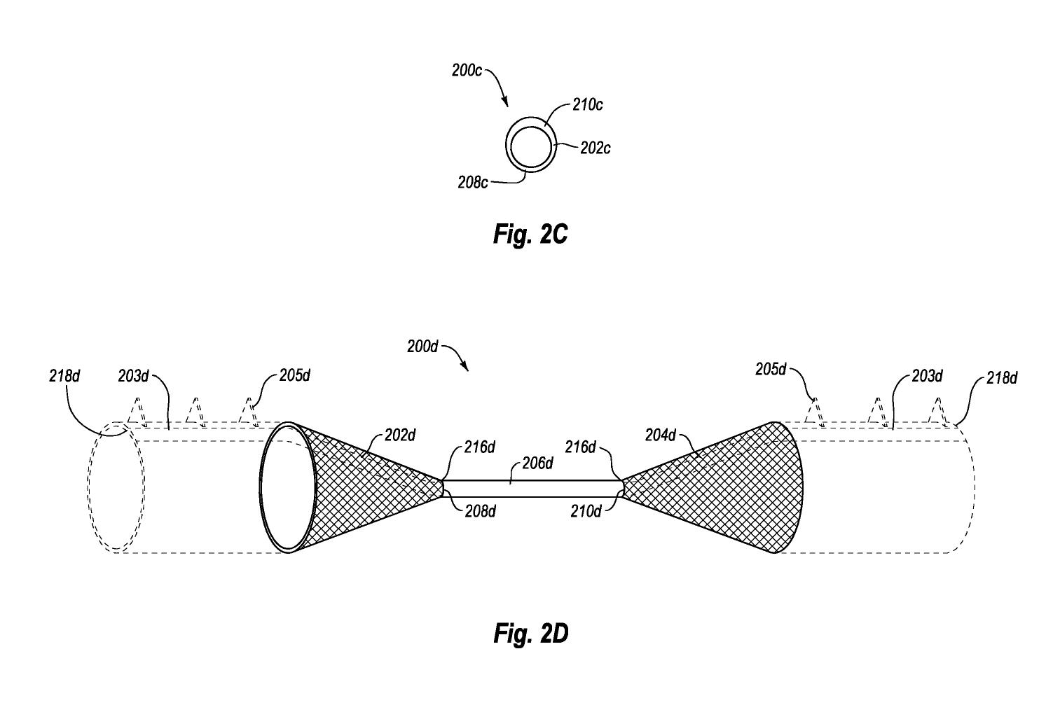

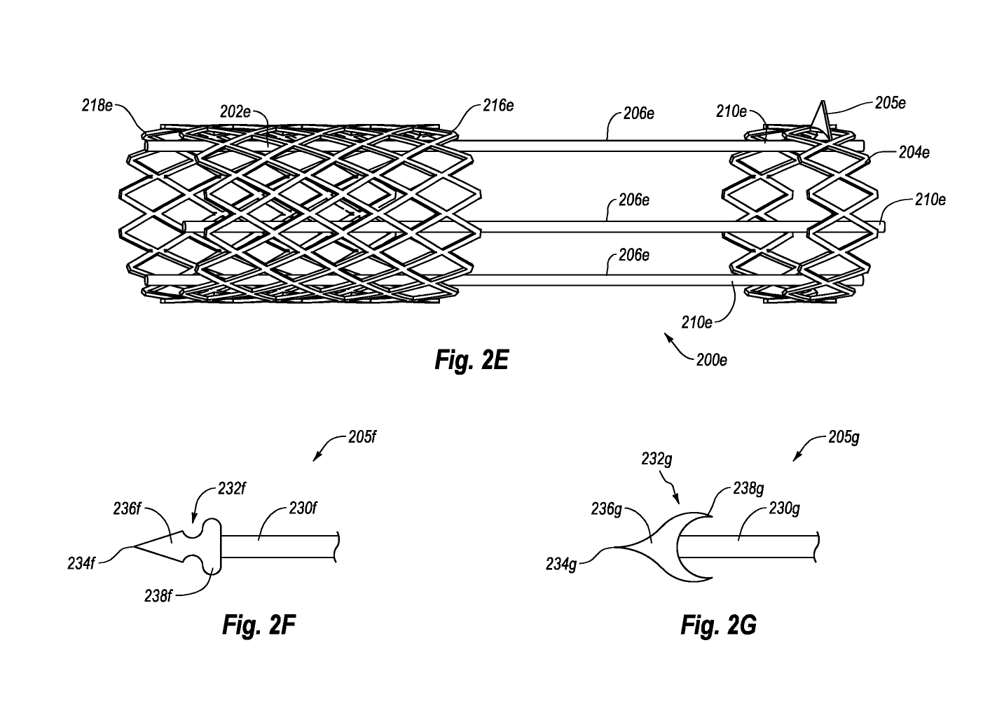

Referring now to FIGS. 2A-2G. FIGS. 2A-2E illustrate different embodiments of the intravascular cardiac restraining implants 200a-200e having different types of anchors as well as different configurations of elongate members. FIGS. 2F and 2G illustrate different embodiments of protruding members that can pierce at least part way through a cardiac vein and anchor the intravascular cardiac restraining implants 200a-200e into the myocardium. Any of the illustrated embodiments or components thereof can be interchanged with any of the other illustrated or described embodiments.

FIG. 2A illustrates a flattened view of the interior surface of a stent-like intravascular cardiac restraining implant 200a having a first stent anchor 202a linked to a second stent anchor 204a through one or more elongate members 206a. By way of example and not limitation, stents that can be useful as anchors can be found in U.S. Pat. Nos. 6,602,285, 7,128,756, and 8,187,324 the entireties of which are incorporated herein by specific reference. Any stent configuration can be used as an anchor.

Each of the first stent anchor 202a and the second stent anchor 204a include an interior end 216a and an exterior end 218a. In the illustrated embodiment, each of the elongate members 206a extend all the way to the outside ends 218a of the first stent anchor 202a and the second stent anchor 204a. This arrangement can increase the torsional rigidity of the stent-like intravascular cardiac restraining implant 200a. Alternatively, the elongate members 206a may be configured so that they do not extend all the way to the outside ends 218a of the first stent anchor 202a and the second stent anchor 204a. Such an arrangement may be used to selectively tailor the torsional rigidity of the stent-like intravascular cardiac restraining implant 200a. Each of the elongate members 206a may be coupled to the first stent anchor 202a and the second stent anchor 204a through a number of couplings, welds, and the like. While not shown, a brace spacer may also be included.

The first and second stent anchors 202a and 204a are configured to be delivered and deployed into a body lumen in much the way stents are configured. The stent anchors 202a and 204a can be expanded and anchored to a vessel tissue independently or at the same time. The elongate members 206a can be elongated during delivery and/or deployment of the implant 200a, or deployed in a configuration that automatically or selectively applies the cinching force to the stent anchors 202a and 204a. The stent anchors 202a and 204a can have any stent configuration.

FIG. 2B illustrates flattened view of the interior surface of an alternative embodiment of a stent-like intravascular cardiac restraining implant 200b having a first stent anchor 202b and a second stent anchor 204b. Each of the first stent anchor 202b and the second stent anchor 204b include an interior end 216a and an exterior end 218a. In the illustrated embodiment, each of the elongate members 206a extend all the way to the outside ends 218a of the first stent anchor 202a and the second stent anchor 204a.

In contrast to the stent-like intravascular cardiac restraining implant 200a shown in FIG. 2A, the first and second stent anchors 202b and 204b have an altered distribution of struts 220, which creates a series of large openings 222 in one region 208b of the first and second stent anchors 202b and 204b and a series of smaller openings 224 in another region 210b of the first and second stent anchors 202b and 204b. Such an irregular distribution of struts is an example of how the flex modulus of the stent-like intravascular cardiac restraining implant 200b can be altered of changed to achieve specific or desired characteristics. For example, region 208b will be less rigid than region 210b.

FIG. 2C illustrates an end view of another embodiment of a stent-like intravascular cardiac restraining implant 200c that can also exhibit altered or changed flexural modulus. In the embodiment illustrated in FIG. 2C, the illustrated stent anchor 202c includes a thinner material region 208c and a thicker material region 210c. Such an irregular distribution of material (e.g., nickel-titanium alloy) will cause such a stent anchor 202c to exhibit greater flexibility in the thinner material region 208c and greater rigidity in the thicker material region 210c.

Such altered flexural moduli of the stent-like intravascular cardiac restraining implants 200b and 200c may, for example, allow the implants 200b and 200c to better conform to the curvature of a patient's heart or the curvature of a cardiac vein at a site of implantation and to aid in the desired delivery orientation of any protruding members (protruding members will be discussed later), such that any protruding members are oriented into the heart tissue and not into the free wall of the vessel. Likewise, such an altered flexural modulus may allow the implants 200b and 200c to better flex and deform in response to normal contractile movement of the heart while simultaneously allowing sufficient rigidity for the implants 200b and 200c to scaffold, reinforce, or reshape diseased or damaged tissues of the heart.

FIG. 2D illustrates a vascular filter-like intravascular cardiac restraining implant 200d having a first filter anchor 202d linked to a second filter anchor 204d through one or more elongate members 206d. The elongate members 206d are each coupled to the first filter anchor 202d through a first coupling 208d and coupled to the second filter anchor 204d through a second coupling 210d. While not shown, a brace spacer may also be included. The first and second filter anchors 202d and 204d are configured to be delivered and deployed into a body lumen in much the way vascular filters are configured. The filter anchors 202d and 204d can be expanded and anchored to a vessel tissue independently or at the same time. In some embodiments, the elongate member 206d can be elongated during delivery and/or deployment of the implant 200d, or deployed in a configuration that automatically or selectively applies the cinching force to the filter anchors 202d and 204d. The filter anchors 202d and 204d can have any type of vascular filter configuration.

Optionally, the implant 200d can also include tubular anchors 203d coupled to the first and second tissue anchors 202d and 204d. The tubular anchors 203d can be configured similarly to a stent, and can have a length sufficient to provide an anchoring feature with improved tissue anchoring and increased anchoring surface area.

Optionally, the implant 200d as well as other implant and/or anchor embodiments can include protruding members 205d extending from one side of the intravascular cardiac restraining implant. The protruding members 205d are selectively positioned such that they can pierce at least part way through a coronary vein at a site of implantation to anchor the implant to the myocardial tissue surrounding the implant 200d for improved anchoring. Preferably, the protruding members 205d should have an overall length sufficient to penetrate the vessel wall and to project a significant distance into the underlying tissue (i.e., the myocardium) to provide support so that the implant 200d can support and or reshape the underlying tissue. Such improved anchoring can, for example, allow the implant 200d to anchor more firmly into the coronary vein to more firmly reinforce and/or reshape the cardiac tissue and avoid slipping in response to contractile movement of the heart.

FIG. 2E illustrates a collapsible intravascular cardiac restraining implant 200e having a first anchor 202e (e.g., configured similarly to a stent) linked to a second anchor 204e. The second anchor 204e can be circular or oval as well as have tissue protruding members 205e so as to be capable of expanding and/or anchoring to a tissue. The second anchor can be configured as an end member, and may have a stent-like or filter-like configuration. In the illustrated embodiment, the second anchor 204e includes at least one protruding member 205e that is positioned to anchor into the myocardial tissue surrounding a coronary vein at a site of implantation. The second anchor 204e can be coupled to the elongate member 206e through a coupling 210c. One or more elongate members 206e can extend to the exterior end 218e of the first anchor 202e. The one or more elongate members 206e can be coupled to the first anchor 202e through a number of couplings, welds, and the like. While not shown, a brace spacer may also be included.

Referring now to FIGS. 2F and 2G, alternative embodiments of protruding members 205f and 205g are illustrated. FIG. 2F illustrates an arrow-like protruding member 205f and FIG. 2G illustrates a barb-like protruding member 205g. The arrow-like protruding member 205f includes a shaft portion 230f and a tip portion 232f. The tip portion 232f includes a sharpened distal portion 234f, a body portion 236f, and a widened proximal portion 238f adjacent to the shaft 230f. The barb-like protruding member 205g includes a shaft portion 230g and a tip portion 232g. The tip portion 232g includes a sharpened distal portion 234g, a body portion 236g, and a barbed proximal portion 238g adjacent to the shaft 230g. When the arrow-like protruding member 205f or the barb-like protruding member 205g is pierced through a coronary vein at a site of implantation, the widened proximal portion 238f or the barbed proximal portion 238g can act to inhibit the protruding members 205f 205g from being withdrawn from or pulled out of the muscular myocardial tissue adjacent to the coronary vein.

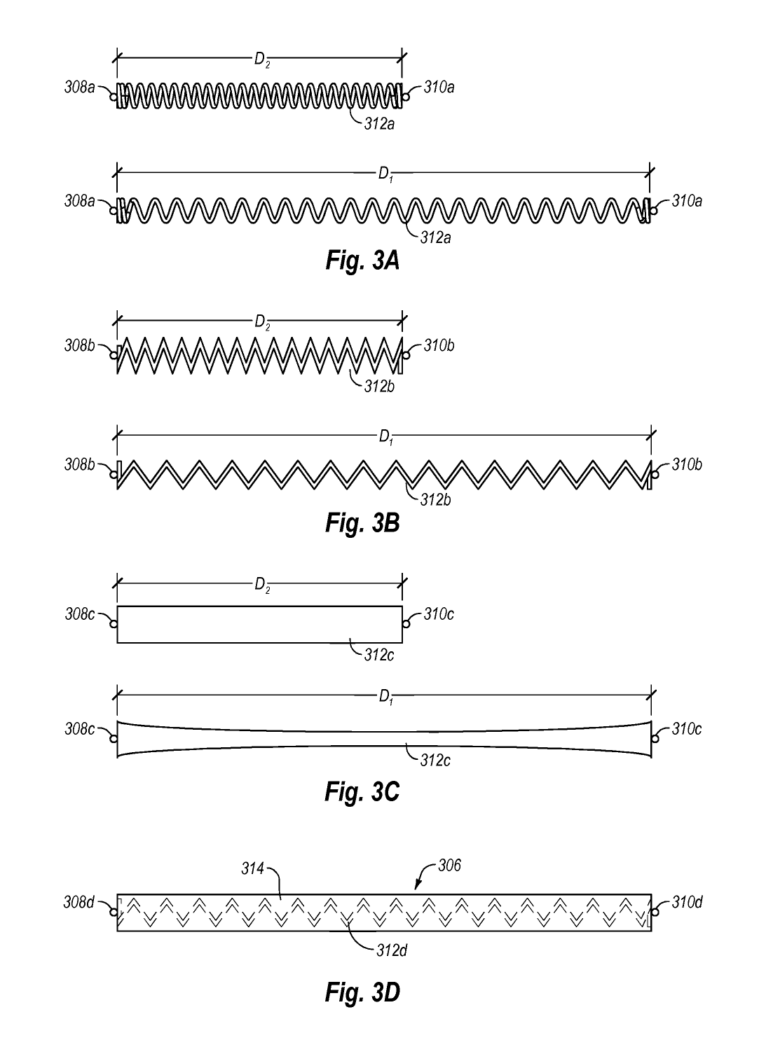

FIGS. 3A-3D show different embodiments of tissue cinching members 306 (e.g., springs or biasing members) in accordance with the present invention. The tissue cinching members 306 can include a tension member 312 configured as described with respect to FIGS. 1A-1C. Each tension member 312 is configured to apply tension to the anchors so as to provide a cinching force to draw the anchors toward each other or provide tension to tissues anchored by the anchors.

FIG. 3A shows a spring tension member 312a that has spring-like or coil-like configuration that has an elongated dimension D1 and a shorter contracted dimension D2. The spring tension member 312a can be coupled to anchors as described herein.

FIG. 3B shows a wave tension member 312b that has wave, zig-zag or sharp sine wave configuration that has an elongated dimension D1 and a shorter contracted dimension D2. The wave tension member 312b can be coupled to anchors as described herein.

FIG. 3C shows an elastic tension member 312a that has an elastic-like configuration that can be stretched to an elongated dimension D1 and retracted back to a shorter contracted dimension D2. The elastic tension member 312c can be coupled to anchors as described herein.

FIG. 3D shows a tissue cinching member 306d having a wave tension member 312d imbedded in a biodegradable non-tension member 314d. An implant having such a tissue cinching member 306d can be implanted and as the biodegradable non-tension member 314d degrades, the wave tension member 312b can impart the cinching force, and even increase the cinching force as the non-tension member 314d degrades. This type of tissue cinching member 306d allows for the implant to be implanted without stretching or elongating the tension member because the tension member has already been pre-stretched or elongated before being embedded in the biodegradable non-tension member 314d.

Description of additional embodiments of elongate members that can be selectively shortened or elongated to either shape the implant (e.g., form a selected curvature) or cinch a tissue at a site of implantation can be found, for example, in U.S. Pat. No. 7,485,143, the entirety of which is incorporated herein by reference.

### III. Methods of Treating Heart Disease Using an Intravascular Cardiac Restraining Implant

Generally, the intravascular cardiac restraining implant of the present invention can be delivered into a body of a subject by any method known or developed. For example, the method of using catheters to percutaneously deploy self-expandable or balloon-expandable stents can be employed.

In one embodiment, the intravascular cardiac restraining implant can be configured for use in a body lumen, such as, but not limited to, a coronary vein (e.g., a coronary sinus). As such, the present invention includes a method of delivering an intravascular cardiac restraining implant into a coronary vein of a subject. Similar methods to those recited herein can be applied to deliver the implant to a body cavity, organ, or other non-lumen body feature.

In one embodiment, a method for treating a diseased, weakened or distended portion of a patient's heart is disclosed. The method includes (1) accessing a coronary vein of the patient's heart percutaneously, (2) positioning an intravascular cardiac restraining implant across at least a portion of the diseased, weakened or distended portion of the patient's heart via the coronary vein, and (3) deploying the intravascular cardiac restraining implant in the coronary vein of the patient's heart for reinforcing or reshaping the diseased, weakened or distended portion of the patient's heart. In one embodiment, the coronary vein includes a coronary sinus.

In another embodiment, a method for treating a diseased heart is disclosed. The method includes (1) providing an intravascular cardiac restraining implant as illustrated in one or more embodiments described herein, (2) percutaneously delivering the implant to a weakened portion of the diseased heart, and (3) anchoring the first tissue and second tissue anchors in a coronary vein such that the first and second tissue anchors and the at least one elongate member span the weakened portion of the heart for remodeling the heart.

In yet another embodiment, a method is disclosed for treating heart failure by providing a support for a diseased, weakened, distended or misshapen portion of a patient's heart. The method includes, (1) percutaneously positioning an intravascular cardiac restraining implant in a coronary vein across at least a portion of the diseased, weakened, distended or misshapen portion of the patient's heart, wherein the intravascular cardiac restraining implant includes the features of intravascular cardiac restraining implant illustrated in one or more embodiments described herein, (2) deploying the intravascular cardiac restraining implant in the coronary vein of the patient's heart for reinforcing or reshaping the diseased, weakened, distended or misshapen portion of the patient's heart, and (3) piercing the coronary vein with the at least one protruding member and anchoring the protruding member into a heart muscle or connective tissue portion adjacent to the coronary vein.

The methods described herein include reinforcing or reshaping diseased, weakened or distended portion of the heart. In one embodiment, the reinforcing or reshaping includes reducing a volume of the heart. As explained in greater detail elsewhere herein, one typical consequence of heart disease and loss of heart function in enlargement of the heart (i.e., megalocardia). The implants and methods described herein can be used to reduce the volume of the heart and counteract the effects of heart disease and megalocardia.

In another embodiment, the reinforcing or reshaping includes reducing distention or bulging of the heart in the vicinity of the intravascular cardiac restraining implant. As explained in greater detail elsewhere herein, one typical consequence of heart disease (e.g., myocardial infarction or congestive heart failure) is the at least partial loss of tissue integrity of the heart. Because of the internal pressures in the heart required to effectively pump blood, such a loss of tissue integrity can lead to bulging and/or distention of the heart muscle. The implants and methods described herein can be used to reinforce or reshape the heart to reduce the tendency of the heart to bulge or distend.

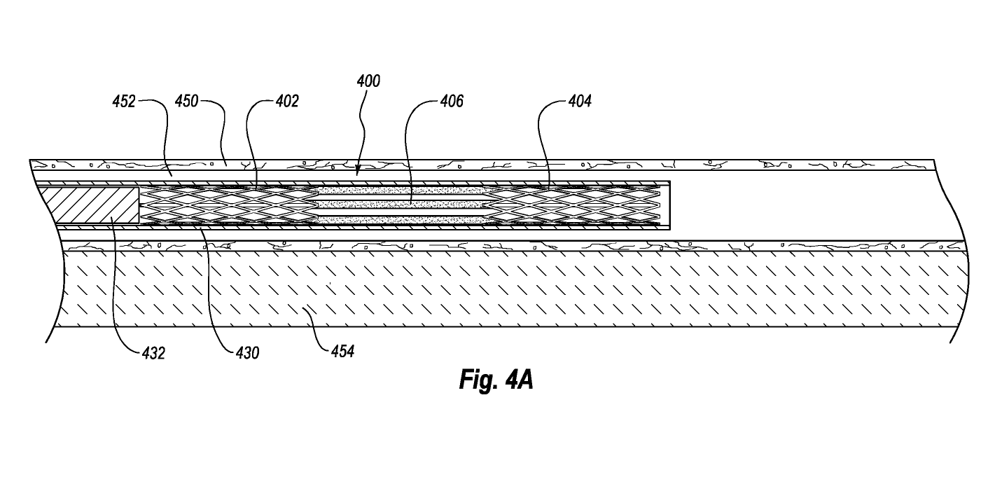

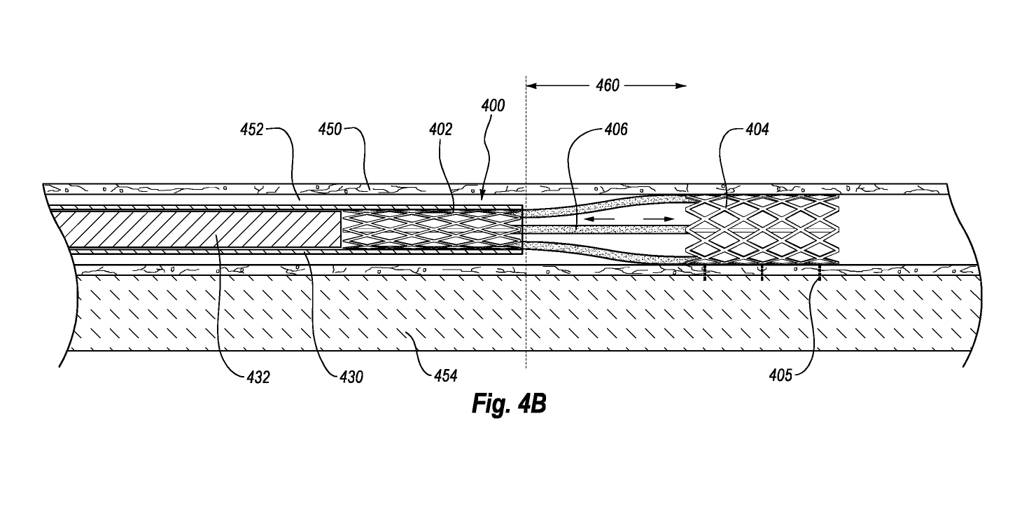

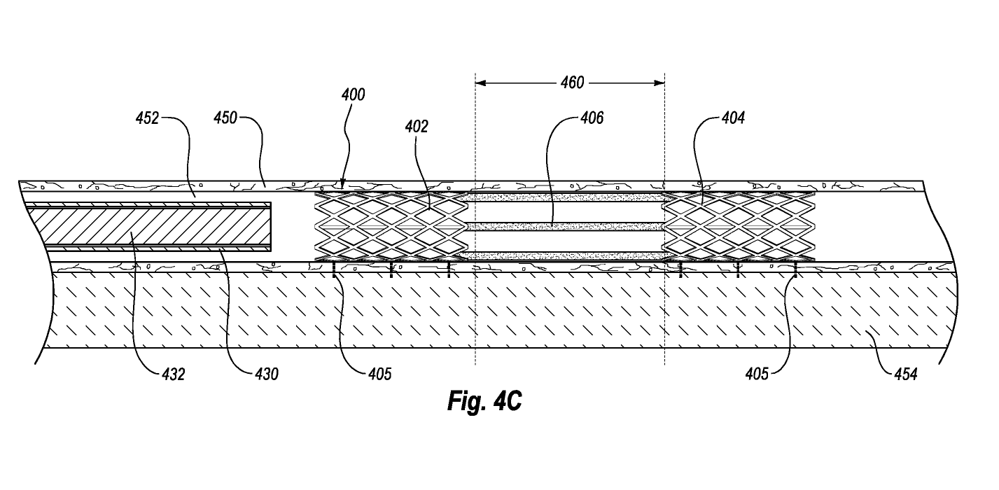

FIGS. 4A-4C are side views illustrating an embodiment of an intravascular cardiac restraining implant 400 and methods of deploying such an intravascular cardiac restraining implant 400 into a blood vessel 450 (e.g., a coronary vein or a coronary sinus) that includes an internal lumen 452 that can receive the intravascular cardiac restraining implant 400. The implant 400 can be configured substantially as shown in other figures provided herewith. The implant 400 can include a first tissue anchor 402 linked to a second tissue anchor 404 through an elongate member 406. The implant 400 can be in a delivery configuration, where as shown the tissue anchors 402 and 404, which are configured like stents, can be collapsed and retained within a delivery device 430, such as a delivery catheter.

FIG. 4A is a schematic representation illustrating a delivery device 430 having the intravascular cardiac restraining implant 400 located therein. The delivery device 430 can be delivered percutaneously into a blood vessel 450 associated with tissue to be reinforced or reshaped. In some instances the vessel 450 itself may need to be reinforced or reshaped. In other instances, the tissue 454 surrounding the blood vessel 450 may need to be reinforced or reshaped. The delivery device 430 can be configured as an implant delivery catheter for delivering an intravascular cardiac restraining implant 400 that is retained by the delivery device 430 in a delivery orientation (e.g., radially compressed). The delivery device 430 can include a deployment member 432 that is configured to push the implant 400 from the delivery device 430. Accordingly, the delivery device 400 is substantially tubular and configured similarly as any delivery catheter member. The deployment member 432 can be configured to be longitudinally stiff with sufficient dimensions to push the implant from the delivery device 400.

While not shown, the delivery device 430 can be a catheter and operated similarly to any method of delivering other implants into a body lumen. As such, an insertion site (not shown) is formed through the skin (not shown) that traverses into a blood vessel at a site remote from the site of implantation. A guidewire (not shown) may then be inserted through the insertion site, through the body lumen 450, to the delivery site. A catheter (not shown) is then inserted into the body lumen 450 to the delivery site over the guidewire, and the guidewire is optionally extracted. The delivery catheter 430 is then inserted through the catheter (not shown) until reaching the delivery site and the catheter is withdrawn.

Optionally, the catheter is the delivery catheter 430, and in this instance, the delivery catheter 430 is retained at the delivery site and the implant 400 is delivered to the delivery site through the delivery catheter 430. A deployment member 432 (pushing member) can be used to push the implant 400 from the delivery catheter 430 for deployment.

FIG. 4B illustrates the implant 400 being deployed within the body lumen 450. As shown, the second anchor 404 has been pushed from the delivery catheter 430 by the deployment member 432 such that the second anchor 404 expands so as to contact the body lumen 450 and anchor itself thereto. Alternatively, the implant 400 can be deployed from the delivery catheter by retracting a restraining sheath. The intravascular cardiac restraining implant 400 is positioned in the body lumen 450 such that the elongate members 406 of the implant span a region 460 of diseased or weakened tissue. Optionally, the second anchor 404 can include protruding members 404, which are shown by the dashed lines, that can pierce at least partially through the body lumen 450 and anchor into the tissue 454 (e.g., myocardial tissue) adjacent to the body lumen 450.

Also, as shown in FIG. 4B the elongate member 406 may be elongated or stretched by withdrawing the delivery device 430 so that the elongate member 406 can apply a cinching force to the implanted second anchor 404. Alternatively, the elongate member 406 may not be elongated or stretched as it may have previously been elongated or stretched and held in the elongated orientation by a brace spacer or an elongate member 406 that includes a tension member and a non-tension member as described.

FIG. 4C shows the first tissue anchor 402 being deployed. As such, the deployment member 432 can push or otherwise deploy the first tissue anchor 402 from the delivery catheter 430. Upon release from the delivery catheter 430, the first tissue anchor 402 can expand similar to a stent to anchor to the body lumen 450. Optionally, the first tissue anchor 402 can include protruding members 405, which are shown by the dashed lines.

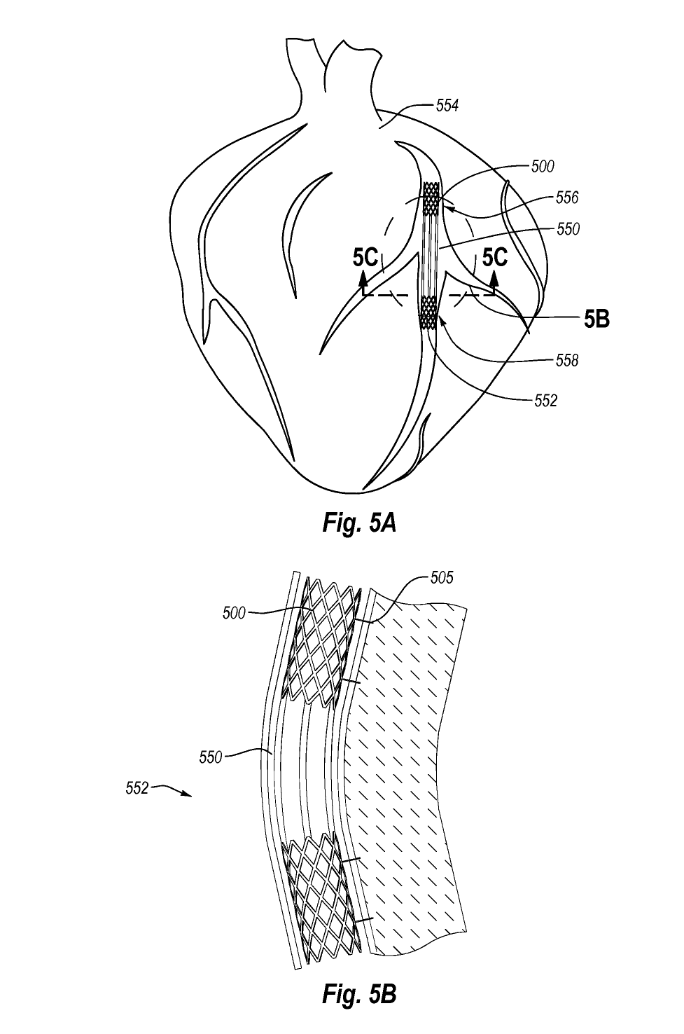

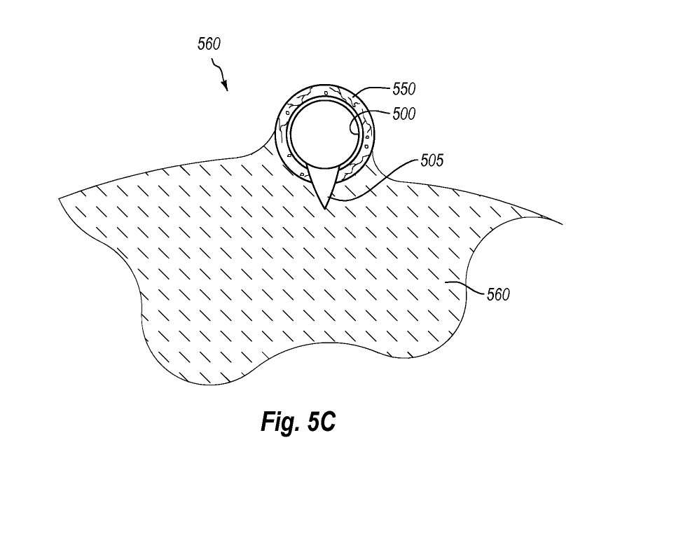

In one embodiment as shown in FIGS. 5A-5C, the intravascular cardiac restraining implant 500 can be anchored to two tissue portions associated with the heart 554. In one aspect, the two tissue portions 556 and 558 of the heart 554 are associated with a cardiac blood vessel 550 (e.g., a coronary sinus), such that the tissue implant 500 is anchored within the cardiac blood vessel 550 to reinforce or reshape the heart 554. The region bound by the dotted oval 552 can define a region of heart tissue that is diseased or damaged, such as from congestive heart failure, that is need of being reinforced and/or reshaped. The implant 500 can be anchored in the blood vessel 550 such that the implant spans the diseased tissue in the diseased tissue region 552.

FIG. 5B illustrates a longitudinal side view of the implant 500 implanted in the cardiac blood vessel 550. The implant 500 includes protruding members that can anchor into the tissue surrounding the site of implantation.

As can be seen in FIG. 5B, the heart is curved and, as a consequence, the implant is also curved. In one embodiment, the intravascular cardiac restraining implant 500 has a size and curvature configured to allow the intravascular cardiac restraining implant to conform to a curvature of the heart at a site of implantation 552. In one embodiment, the size and curvature of the implant 500 can reflect the size and curvature of the heart in the region of implantation at the time of implantation 552. That is, the implant 500 can have the size and shape of the diseased heart. In another embodiment, the size and curvature of the intravascular cardiac restraining implant 500 can be selected to reflect a size and curvature of a coronary vein of a healthy heart at the site of implantation 552. In such an embodiment, the implant 500 can be used to reshape at least a portion of the heart such that it has the size and shape of a healthy heart. In one embodiment, the implant 500 may be manufactured with a preselected curvature. In another embodiment, the implant 500 may be manufactured without a curvature so that it is substantially linear when it is unconstrained. Additionally of in lieu of a manufactured shape, the intravascular cardiac restraining implant 500 may be user shapeable.

The curvature of the heart and the implant shown in FIG. 5B represents a relatively simple arc. Nevertheless, the curvature of the intravascular cardiac restraining implant 500 may include a compound (i.e., complex) curvature.

FIG. 5C shows a cross section 560 of the heart 554 and vessel 550 with the implant 500 disposed therein with a protruding member 505 anchoring into the tissue of the heart adjacent to the blood vessel 550.

In one embodiment, the present invention can include a method of extracting the implant from the body of a subject, such as from a body lumen. The extraction method can include: inserting an implant-extracting medical device into the body lumen so as to come into contact with the implant, which implant extracting medical device can be configured as a catheter; engaging the implant-extracting medical device with the implant; radially compressing the implant so as to have a reduced dimension with a cross section that is smaller than the body lumen; and retrieving the implant from the desired deployment site within the body lumen of the subject. Optionally, the implant can be received into the implant-extracting medical device, which can be substantially similar to a catheter.

While the disclosure of this document relates in many instances to an intraluminal intravascular cardiac restraining implant, the anchors could also be used for anchoring into any type of tissue in any location and drawing the two anchored tissues toward each other. In some instance one of the anchored tissues will be substantially immobile such that the other tissue will be drawn toward the substantially immobile tissue. In other instances, the tissues may be substantially immobile such that the intravascular cardiac restraining implant provides a cinching force to aid in retaining the tissues where they are located in a body.

The present invention may be configured in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope. All references recited herein are incorporated herein by specific reference.

1. A method for treating a distended portion of a patient's heart, the method comprising:
accessing a coronary vein of the patient's heart percutaneously;
shaping an intravascular cardiac restraining implant;
following shaping, positioning an intravascular cardiac restraining implant across at least a portion of the distended portion of the patient's heart via the coronary vein; and
deploying the intravascular cardiac restraining implant in the coronary vein of the patient's heart for reinforcing or reshaping the distended portion of the patient's heart.

2. (canceled)

3. The method of claim 1, wherein the coronary vein includes a coronary sinus.

4. The method of claim 1, wherein the reinforcing or reshaping includes reducing a volume of the heart.

5. The method of claim 1, wherein the reinforcing or reshaping includes reducing distention of the heart in the vicinity of the intravascular cardiac restraining implant.

6. The method of claim 1, wherein the intravascular cardiac restraining implant has a size and curvature configured to allow the intravascular cardiac restraining implant to conform to a curvature of the coronary vein at a site of implantation.

7. The method of claim 6, wherein the size and curvature of the intravascular cardiac restraining implant are selected to reflect a size and curvature of a coronary vein of a healthy heart at the site of implantation.

8. The method of claim 6, wherein the curvature of the intravascular cardiac restraining implant includes a compound curvature.

9. (canceled)

10. The method of claim 1, wherein the intravascular cardiac restraining implant includes:
a first tissue anchor configured for implantation in a first region of the coronary vein;
a second tissue anchor configured for implantation in a second region of the coronary vein; and
at least one elongate member coupled to the first tissue anchor and the second tissue anchor.

11. The method of claim 10, wherein the first and second tissue anchors are configured as lumen endoprostheses.

12. The method of claim 1, wherein at least one portion of the intravascular cardiac restraining implant is fabricated from a shape memory material.

13. The method of claim 12, wherein the shape memory material includes a nickel-titanium alloy.

14. A method for treating a distended portion of a diseased heart, the method comprising:
providing an intravascular cardiac restraining implant, including:
a first tissue anchor configured for implantation in a first region of a coronary vein, the first tissue anchor being a filter;
a second tissue anchor configured for implantation in a second region of the coronary vein, the second tissue anchor being a filter; and
at least one elongate member coupled to the first tissue anchor and the second anchor,
wherein the intravascular cardiac restraining implant has a size and curvature selected to allow the medical device to conform to a size and curvature of a portion of the diseased heart;
percutaneously delivering the implant to the distended portion of the diseased heart; and
anchoring the first tissue and second tissue anchors in a coronary vein such that the first and second tissue anchors and the at least one elongate member span the distended portion of the heart for remodeling the heart and filtering blood flowing through the first tissue anchor and the second tissue anchor.

15. The method of claim 14, wherein at least one of the first tissue anchor or the second tissue anchor includes portions having a variable flexural modulus.

16. (canceled)

17. The method of claim 14, wherein at least one of the first tissue anchor or the second tissue anchor is self-expanding.

18. The method of claim 14, wherein at least one of the first tissue anchor or the second tissue anchor is balloon expandable.

19. (canceled)

20. The method of claim 14, wherein the lumen endoprostheses have a conical shape.

21. (canceled)

22. The method of claim 14, each of the first tissue anchor and the second tissue anchor having an interior end and an exterior end, wherein the interior ends are oriented toward one another and the exterior ends are oriented away from one another, and wherein the at least one elongate member extends substantially from the exterior end of the first tissue anchor to the opposite exterior end of the second tissue anchor.

23. The method of claim 14, wherein the at least one elongate member includes one or more tension members configured to apply a cinching force to the first and second tissue anchors so as to draw the first and second tissue anchors toward one another.

24. The method of claim 23, wherein the one or more tension members are configured as a spring, coil, waveform, zig-zag, elastic, worm drive, ratchet drive, arcuate member, cinchable member, or a combination thereof.

25. The method of claim 14, further comprising one or more extension spacers and/or brace spacers removably located between the first and second tissue anchors.

26. The method of claim 25, wherein the one or more extension spacers and/or brace spacers are biodegradable.

27. The method of claim 14, wherein
at least one of the first tissue anchor or the second tissue anchor has at least one protruding member extending from one side of the intravascular cardiac restraining implant; and
the method further comprising piercing the coronary vein with the at least one protruding member and anchoring the protruding member into a heart muscle or connective tissue portion adjacent to the coronary vein.

28. The method of claim 27, wherein the least one protruding member is selected from a group consisting of hooks, barbs, screws, corkscrews, coils, helices, and flanges to anchor at least one of the first tissue anchor or the second tissue anchor to the heart muscle or connective tissue portion adjacent to the coronary vein.

29. A method for treating heart failure by providing support for a distended portion of a patient's heart, the method comprising:
percutaneously positioning an intravascular cardiac restraining implant in a coronary vein across at least a portion of the distended portion of the patient's heart,
wherein the intravascular cardiac restraining implant includes:
a first tissue anchor configured for implantation in a first region of the cardiac vein;
a second tissue anchor configured for implantation in a second region of the cardiac vein;
at least one elongate member coupled to the first tissue anchor and the second anchor, the at least one elongate member being a tissue cinching member having a tension member imbedded in a biodegradable non-tension member; and
at least one of the first tissue anchor or the second tissue anchor having at least one protruding member extending from one side of the intravascular cardiac restraining implant;
deploying the intravascular cardiac restraining implant in the coronary vein of the patient's heart for reinforcing or reshaping the distended portion of the patient's heart; and
piercing the coronary vein with the at least one protruding member and anchoring the protruding member into a heart muscle or connective tissue portion adjacent to the coronary vein.

30. The method of claim 29, wherein the at least one elongate member is a tissue cinching member configured to draw a first portion of the patient's heart toward a second portion of the patient's heart.

31. (canceled)

32. (canceled)

33. (canceled)

34. The method of claim 29, further comprising allowing the biodegradable non-tension member to biodegrade after deploying so as to draw the first tissue portion toward the second tissue portion.

35. The method of claim 29, wherein the at least one elongate member is a flexurally stiff member configured to resist distension caused by intracardiac pressure or growth of the heart.

36. The method of claim 29, wherein at least one of the first tissue anchor or the second tissue anchor includes portions having a variable flexural modulus.

37. The method of claim 36, further comprising deploying the intravascular cardiac restraining implant in the coronary vein is an orientation such that the variable flexural modulus allows the intravascular cardiac restraining implant to conform to the shape and curvature of the heart while maintaining flexural stiffness to resist distension caused by intracardiac pressure or growth of the heart.
